(19) United States
(12) Patent Application Publication
(10) Pub. No.: US 2006/0148686 A1
(43) Pub. Date: Jul. 6, 2006

(54) Ophthalmic compositions comprising steroid and cyclosporine for dry eye therapy

(75) Inventors: Erning Xia, Penfield, NY (US); Zhenze Hu, Pittsford, NY (US); Praveen Tyle, Pittsford, NY (US); Stephen Bartels, Pittsford, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/311,534

(22) Filed: Dec. 19, 2005

Related U.S. Application Data
(60) Provisional application No. 60/640,659, filed on Dec. 30, 2004.

(51) Int. Cl. A61K 38/13; A61K 31/573

(52) U.S. Cl. 514/11; 514/171

(57) ABSTRACT

The invention comprises a method of treating dry eye in a patient in need of such treatment. The treatment comprises administering to a patient in need thereof an ophthalmic pharmaceutical composition comprising a corticosteroid and cyclosporine. In a further embodiment of the invention the corticosteroid is loteprednol etabonate. In yet a further embodiment of the invention the cyclosporine is present in the ophthalmic composition as either a natural or synthetic cyclosporine.

### CROSS REFERENCE

This application claims the benefit of Provisional Patent Application No. 60/640,659 filed Dec. 30, 2004 and is incorporated herein by reference.

### BACKGROUND

The use of cyclosporine for ophthalmic indications is known. For example, U.S. Pat. No. 4,649,047 discloses a method for the treatment of either phacoanaphylactic endophthalmitis or uveitis by administering at least one cyclosporin topically to the eyes. Topical application of cyclosporin provides cyclosporin to the anterior chamber, the posterior chamber and the vitreous body of the eye. U.S. Pat. No. 4,838,342 discloses a method of treating an aqueous-deficient dry eye state in a patient suffering therefrom, which method includes the step of administering cyclosporin topically to the patient's eye. The cyclosporin is administered as a solution, suspension or ointment in a pharmaceutically acceptable excipient. U.S. Pat. No. 5,479,979 discloses pharmaceutical compositions in the form of a nonirritating emulsion which includes at least one cyclosporin in admixture with a higher fatty acid glyceride and polysorbate 80. More particularly, the cyclosporin may be cyclosporin A and the higher fatty acid glyceride may be castor oil. The latter patent is identified on the package insert as claiming at least part of the product RESTASIS, an ophthalmic emulsion indicated to increase tear production in patients whose tear production is presumed to be suppressed due to ocular inflammation associated with keratoconjunctivitis sicca.

It has been noted that the use of anti inflammatory drugs at the same time as using RESTASIS will interfere with the desired effect of increased tear production. We believe that the combination of anti inflammatory drugs such as steroids with cyclosporine provides an effective treatment for dry eye and restores tears correct in composition and sufficient in volume.

In addition, it has been discovered that such compositions are also useful for treating anterior segment inflammatory eye diseases including dry eye.

Future treatments or therapies for dry eye will or already focus upon actively suppressing inflammatory mechanisms rather than passively lubricating the ocular surface. Artificial tears may be particularly useful when they are specially formulated to correct and balance the hyperosmolarity of the tear film. This may serve to interrupt the inflammatory cycle enough to allow at least temporary ocular surface healing.

The combination of corticosteroid (such as Loteprednol etabonate) with immunosuppressive agents (such as Cyclosporine) can modulate or interrupt the inflammatory-based pathogenisis of some types of dry eye disease and offer significant advantages over traditional dry eye therapies.

Steroids appear to have an effect on nearly every aspect of the immune system. They inhibit:

(1) both migration of Eosinophils into the extra cellular space and the adherence to the vascular endothelium at the site of ocular tissue injury,

(2) macrophage access to the site of inflammation and interfere with lymphocyte activity as well as decreasing the number of B and T lymphocytes,

(3) phospholipase A2, which prevents biosynthesis of arachidonic acid and subsequent formation of prostacyclin, thromboxane A, prostaglandins and leukotrienes.

The addition of cyclosporine to a dry eye composition enhances the anti-inflammatory effect. Although not wishing to be bound by a particular theory, it is believed that cyclosporine exerts a selective immunosuppressive effect by blocking an early stage activation of cytotoxic T lymphocytes in response to antigens. Cytokines in normal tear fluid inhibit conjunctival epithelial proliferation, promote terminal differentiation, stimulate epithelial membrane mucus production and promote goblet cell suppressed production of soluble mediators of inflammation in the tear film, thereby stimulating tear production. However, there are a number of destructive actions that elevated levels of proinflammatory cytokines would induce and for which the combination product of this invention would provide a treatment. Therefore the inhibitory effect of the combination product of this invention on the negative effects produced by elevated levels of pro-inflammatory cytokines would be desirable.

### DESCRIPTION

The invention comprises a method of treating dry eye in a patient in need of such treatment. The treatment comprises administering to a patient in need thereof an ophthalmic pharmaceutical composition comprising a corticosteroid and cyclosporine. In a further embodiment of the invention the corticosteroid is loteprednol etabonate. In yet a further embodiment of the invention the cyclosporine is present in the ophthalmic composition as either a natural or synthetic cyclosporine. These and further embodiments of the invention will become apparent to those of ordinary skill in the art by the description and examples and the claims appended hereto.

The invention further compromises a method of treating anterior segment eye disease including dry eye.

The corticosteroid useful in the ophthalmic composition may be any that is now known or yet to be discovered. Examples of known corticosteroids include dexamethasone, loteprednol etabonate, etc. Other steroidal compounds such as prednisolone and related compounds and low solubility steroids such as fluocinolone acetonide and related compounds are envisioned as being within the content of the invention disclosed herein as well as anti-inflammatories such as hydrocortisone, hydrocortisone acetate, dexamethasone 21-phosphate, fluocinolone, medrysone, methylprednisolone, prednisolone 21-phosphate, prednisolone acetate, fluorometholone, betamethasone and triamcinolone. Typical concentrations of steroids in the final formulation may range from 0.01 to 2.0 percent by weight.

Cyclosporins which are useful in the practice of the present invention are either natural or synthetic cyclosporin. For example, Cyclosporin A is advantageously used in the practice of the present invention. Other forms of cyclosporins (e.g., analogs and isomers such as Cyclosporins B, C, D, E, and H) may also be used. Mixtures of at least two different cyclosporins may be used. The cyclosporin is advantageously administered topically as an ophthalmic drop (solution or suspension) or ophthalmic ointment containing an effective amount of the cyclosporin. Concentrations of 0.01 to 20 weight percent, preferably 0.1 to 5 weight percent, of a cyclosporin are used. In accordance with the method of the present invention, a cyclosporin is administered topically in any quantity required to provide the degree of treatment needed. For example, 5 microliters to 1 milliliter of a solution, suspension or ointment containing an effective amount of a cyclosporin, such as 0.01 to 20 weight percent, preferably 0.1 to 5 weight percent, of cyclosporin is advantageously used.

In addition to corticosteroid and cyclosporine, the compositions of the invention herein may also contain ophthalmically acceptable buffers such as Sodium borate/Boric acid. Other ophthalmically acceptable buffers suitable for use in the invention herein will be apparent to one having ordinary skill in the art.

The composition may further comprise ophthalmic demulcents such as any of the following, within the established concentrations for each ingredient: (a) Cellulose derivatives: (1) Carboxymethylcellulose sodium, 0.2 to 2.5 percent, (2) Hydroxyethyl cellulose, 0.2 to 2.5 percent, (3) Hypromellose, 0.2 to 2.5 percent and (4) Methylcellulose, 0.2 to 2.5 percent; (b) Dextran 70, 0.1 percent when used with another polymeric demulcent agent in this section; (c) Gelatin, 0.01 percent; (d) Polyols, liquids such as (1) Glycerin, 0.2 to 1 percent, (2) Polyethylene glycol 300, 0.2 to 1 percent, (3) Polyethylene glycol 400, 0.2 to 1 percent, (4) Polysorbate 80, 0.2 to 1 percent, (5) Propylene glycol, 0.2 to 1 percent, (e) Polyvinyl alcohol, 0.1 to 4 percent and (f) Povidone, 0.1 to 2 percent.

The composition may further comprise balanced salts such as ZnCl₂ and MgCl₂. Other inorganic materials such as HAP (tetrasodium etidronate (Monsanto)), tricalcium phosphate, dicalcium pyrophosphate, tetracalcium phosphate and octacalcium phosphate may also be included.

The composition may further comprise active agents such as any compound, composition of matter, or mixture thereof that can be delivered from the composition of the invention to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; benzodiazepine receptor agonists such as abecarnil; GABA receptor modulators such as baclofen, muscimol and benzodiazepines; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as insulin, growth hormones, insulin related growth factor, heat shock proteins and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; blood flow enhancers; antiasthmatic drugs; antiparasiticagents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors and cytokine protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; svmpathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the formulation and administered using the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

pH adjusting agents such as HCl and NaOH may also be used to adjust the pH of the final composition to between about 4.0 and about 8.5.

Tonicity agents such as those commonly used in the art may be used to bring the osmolality of the final composition to between about 220 and about 400 mOsmo/Kg.

Numerous advantages accrue with the practice of the present invention. The method of the present invention is useful in that it can locally prevent activation of a presystemic response. Topical administration of a cyclosporin and steroid into a patient's tear deficient eye decreases inflammation in the eye. Thus, such treatment further serves to correct corneal and conjunctival disorders exacerbated by tear deficiency and KCS, such as corneal scarring, corneal ulceration, inflammation of the cornea or conjunctiva, filamentary dermatitis, mucopurulent discharge and vascularization of the cornea. Furthermore, cyclosporine directly decreases the immune response of granulation and neovascularization in the cornea.

Compositions according to the invention herein are also useful for treatment of diseases and disorders involving the anterior segment of the eye such as rubeosis iridis, iritis, cyclitis and uveitis. Further objects of this invention, together with additional features contributing thereto and advantages accruing therefrom, will be apparent from the following examples which are intended to illustrate but not limit the invention as defined by the claims contained herein. All values are weight percent unless otherwise specified.

### EXAMPLES

EXAMPLE 1

| Ingredient | gram | |
|---|---|---|
| Phase I | | |
| Carbopol 934P NF | 0.25 | gm |
| Purified Water | 99.75 | gm |
| Phase II | | |
| Propylene Glycol | 5.0 | gm |
| EDTA | 0.1 | mg |
| Loteprednol Etabonate | 50.0 | gm |
| Cyclosporine | 5.0 | gm |

Mix five parts of phase II with twenty parts of phase I for more than 15 minutes and adjust pH to 6.2-6.4 using 1 N NaOH.

EXAMPLE 2

| Ingredient | Grams | |
|---|---|---|
| Phase I | | |
| Carbopol 934P NF | 0.25 | gm |
| Purified Water | 99.75 | gm |
| Phase II | | |
| Propylene Glycol | 3.0 | gm |
| Triacetin | 7.0 | gm |
| Loteprednol Etabonate | 50.0 | gm |
| Cyclosporine | 5.0 | |
| EDTA | 0.1 | mg |

Mix five parts of phase II with twenty parts of phase I for more than 15 minutes and adjust pH to 6.2-6.4 using 1 N NaOH.

EXAMPLE 3

| Ingredient | Grams | |
|---|---|---|
| Phase I | | |
| Carbopol 934P NF | 0.25 | gm |
| Purified Water | 99.75 | gm |
| Phase II | | |
| Propylene Glycol | 7.0 | gm |
| Glycerin | 3.0 | gm |
| Loteprednol Etabonate | 50.0 | gm |
| Cyclosporine | 5.0 | gm |
| HAP (30%) | 0.5 | mg |
| Alexidine 2HCl | 1-2 | ppm |

Mix five parts of phase II with twenty parts of phase I for more than 15 minutes and adjust pH to 6.2-6.4 using 1 N NaOH.

EXAMPLE 4

| Ingredient | % W/W |
|---|---|
| Povidone | 1.00 |
| HAP (30%) | 0.05 |
| Glycerin | 3.00 |
| Propylene Glycol | 3.00 |
| Loteprednol Etabonate | 0.50 |
| Cyclosporine | 0.10 |
| Tyloxapol | 0.25 |
| Alexidine 2HCl | 1-2 ppm |

EXAMPLE 5

| Ingredient | % W/W |
|---|---|
| Povidone | 1.50 |
| HAP (30%) | 0.05 |
| Glycerin | 3.00 |
| Propylene Glycol | 3.00 |
| Loteprednol Etabonate | 0.75 |
| Cyclosporine | 0.10 |
| Tyloxapol | 0.25 |
| Alexidine 2HCl | 1-2 ppm |

EXAMPLE 6

| Ingredient | % W/W |
|---|---|
| CMC (MV) | 0.50 |
| HAP (30%) | 0.05 |
| Glycerin | 3.00 |
| Propylene Glycol | 3.00 |
| Loteprednol Etabonate | 0.75 |
| Cyclosporine | 0.10 |
| Tyloxapol | 0.25 |
| Alexidine 2HCl | 1-2 ppm |

The above examples are intended to illustrate but not limit the invention which is described in the specification and defined by the claims appended here to.

What is claimed is:

1. Ophthalmic pharmaceutical compositions comprising a corticosteroid and a cyclosporin.

2. The composition of claim 1 wherein the corticosteroid is selected from the group consisting of dexamethasone, loteprednol etabonate, prednisolone, fluocinolone acetonide, hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triamcinolone.

3. The composition of claim 1 wherein the cyclosporin is selected from the group consisting of natural and synthetic cyclosporin.

4. The composition of claim 3 wherein the cyclosporin is selected from the group consisting of Cyclosporin A and analogs and isomers thereof such as Cyclosporins B, C, D, E, and H.

5. The composition of claim 1 further comprising a demulcent selected from the group consisting of Cellulose derivatives such as Carboxymethylcellulose sodium, Hydroxyethyl cellulose, Hypromellose, and Methylcellulose; Dextran 70; Gelatin; liquid Polyols such as Glycerin, Polyethylene glycol 300, Polyethylene glycol 400, Polysorbate 80, Propylene glycol, Polyvinyl alcohol, Povidone and mixtures thereof.

6. The composition of claim 1 further comprising inorganic molecules selected from the group consisting of ZnCl₂, MgCl₂, HAP, tricalcium phosphate, dicalcium pyrophosphate, tetracalcium phosphate, octacalcium phosphate and mixtures thereof.

7. The composition of claim 1 wherein the corticosteroid is Loteprednol etabonate and is present in an amount of between about 0.01 wt. % and 10.0 wt. % of the final composition.

8. The composition of claim 1 wherein the corticosteroid is Loteprednol etabonate and is present in an amount of between about 0.01 wt. % and 1.0 wt. % of the final composition.

9. The composition of claim 1 wherein the cyclosporin is present in an amount of between about 0.01 wt. % and 20.0 wt. % of the final composition.

10. The composition of claim 1 wherein the cyclosporin is present in an amount of between about 0.1 wt. % and 5.0 wt. % of the final composition.

11. A method for the treatment of dry eye in a patient in need of such treatment, the treatment comprising administering to a patient in need thereof an effective amount of an ophthalmic pharmaceutical composition comprising a corticosteroid and cyclosporin.

12. The method of claim 11 wherein the corticosteroid is Loteprednol etabonate.

13. The method of claim 11 wherein the cyclosporin is selected from the group consisting of natural and synthetic cyclosporin.

14. The method of claim 11 wherein the cyclosporin is selected from the group consisting of Cyclosporin A and analogs and isomers thereof such as Cyclosporins B, C, D, E, and H.

15. The method of claim 12 wherein the Loteprednol etabonate is present in an amount of between about 0.01 wt. % and 10.0 wt. % of the final composition.

16. The method of claim 13 wherein the Loteprednol etabonate is present in an amount of between about 0.01 wt. % and 1.0 wt. % of the final composition.

17. The method of claim 13 wherein the cyclosporin is present in an amount of between about 0.01 wt. % and 10.0 wt. % of the final composition.

18. The method of claim 13 wherein the cyclosporin is present in an amount of between about 0.1 wt. % and 5.0 wt. % of the final composition.

19. A method for the treatment of diseases and disorders involving the anterior segment of the eye in a patient in need of such treatment, the treatment comprising administering to a patient in need thereof an effective amount of an ophthalmic pharmaceutical composition comprising a corticosteroid and cyclosporin to treat diseases and disorders involving the anterior segment of the eye.

20. The method of claim 19 wherein the diseases and disorders involving the anterior segment of the eye is selected from the group consisting of rubeosis iridis, iritis, cyclitis, uveitis diseases and combinations thereof.

21. The method of claim 19 wherein the corticosteroid is Loteprednol etabonate.

22. The method of claim 19 wherein the cyclosporin is selected from the group consisting of natural and synthetic cyclosporin.

23. The method of claim 19 wherein the cyclosporin is selected from the group consisting of Cyclosporin A and analogs and isomers thereof such as Cyclosporins B, C, D, E, and H.

24. The method of claim 21 wherein the Loteprednol etabonate is present in an amount of between about 0.01 wt. % and 10.0 wt. % of the final composition.

25. The method of claim 21 wherein the Loteprednol etabonate is present in an amount of between about 0.01 wt. % and 1.0 wt. % of the final composition.

26. The method of claim 23 wherein the cyclosporin is present in an amount of between about 0.01 wt. % and 10.0 wt. % of the final composition.

27. The method of claim 23 wherein the cyclosporin is present in an amount of between about 0.1 wt. % and 5.0 wt. % of the final composition.
